(19) United States
(12) Patent Application Publication
(10) Pub. No.: US 2015/0290171 A1
(43) Pub. Date: Oct. 15, 2015

(54) METHODS FOR THE TREATMENT OF BONE LOSS

(71) Applicant: CELGENE CORPORATION, Summit, NJ (US)

(72) Inventors: Mary Adams, Mountain Lakes, NJ (US); Randall Stevens, Plainfield, NJ (US); Peter H. Schafer, Belle Mead, NJ (US)

(21) Appl. No.: 14/441,808

(22) PCT Filed: Nov. 8, 2013
(86) PCT No.: PCT/US2013/069191

§ 371 Date: May 8, 2015

Related U.S. Application Data
(60) Provisional application No. 61/724,749, filed on Nov. 9, 2012.

(51) Int. Cl. A61K 31/4035; A61K 45/06

(52) U.S. Cl. CPC A61K 31/4035; A61K 45/06

(57) ABSTRACT

Provided herein are methods of treating, preventing and/or managing bone loss using (+)-2-[1-(3-ethoxy-4-methoxyphenyl)-2-methanesulfonylethyl]-4-acetylaminoisoindolin-1,3-dione, alone or in combination with other therapeutics.

### 1. CROSS REFERENCE TO RELATED APPLICATIONS

This application claims the benefit of priority to U.S. Provisional Patent Application Ser. No. 61/724,749, filed Nov. 9, 2012, the disclosure of which is incorporated by reference herein in its entirety.

### 2. FIELD

Provided herein are methods of treating, preventing and/or managing loss of bone by the administration of (+)-2-[1-(3-ethoxy-4-methoxyphenyl)-2-methanesulfonylethyl]-4-acetylaminoisoindolin-1,3-dione, alone or in combination with other therapeutics. Also provided herein are pharmaceutical compositions and dosing regimens.

### 3. BACKGROUND

Formation and loss of bones are controlled by several types of bone cells. For example, it is known that focal articular bone loss is mediated by osteoclasts (“OCL”), while osteoblasts (“OBL”) regulate bone remodeling with the ability to produce and mineralize bone matrix. In addition, osteocytes (“OCY”) are also known to be involved in controlling the bone formation. Specifically, it was reported that patients with bone loss (e.g., rheumatoid arthritis patients) have increased expression of sclerostin (“SOST”) in OCY, which suppresses bone formation, while patients with ankylosing spondytilis have reduced expression of the same gene, which may contribute to bone formation.

Bone loss may occur as a primary symptom, or it can be secondary to other underlying disorders. Furthermore, therapeutics that are used to treat certain disorders may cause loss of bone. Loss of bone is thus a serious problem, causing discomfort, fracture and pain, and sometimes immobility in patients suffering from diseases that affect bones. Thus, a need exists for safe and effective treatment for bone loss.

### 4. SUMMARY

Provided herein are methods of treating, managing or preventing loss of bone comprising administering to a patient in need of such treatment, management or prevention a PDE4 inhibitor. In one embodiment, the loss of bone is a primary disorder, e.g., primary type osteoporosis. In another embodiment, the loss of bone is secondary to other underlying disorders, e.g., secondary osteoporosis. In another embodiment, the loss of bone is caused by a therapeutic used for treatment of the underlying disorder, e.g., corticosteroid induced osteoporosis.

In one embodiment, the PDE4 inhibitor is (+)-2-[1-(3-ethoxy-4-methoxyphenyl)-2-methanesulfonylethyl]-4-acetylaminoisoindolin-1,3-dione, which has the following chemical structure:

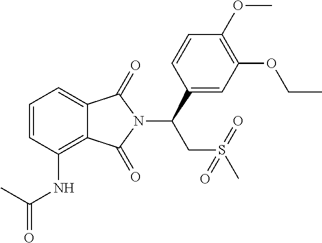 [SMILES: CCOc1cc([C@@H](CS(C)(=O)=O)N2C(=O)c3cccc(NC(C)=O)c3C2=O)ccc1OC]

or a pharmaceutically acceptable salt or solvate (e.g., hydrate) thereof.

In some embodiments, the PDE4 inhibitor is administered in combination with a therapy conventionally used to treat, prevent or manage the loss of bone.

Also provided herein are pharmaceutical compositions, single unit dosage forms, dosing regimens and kits which comprise a PDE4 inhibitor, or a pharmaceutically acceptable salt, solvate, hydrate, clathrate, or prodrug thereof, and a second, or additional, active agent. Second active agents include specific combinations, or “cocktails,” of drugs.

### 5. DETAILED DESCRIPTION

Provided herein are methods of treating, managing or preventing loss of bone comprising administering to a patient in need of such treatment, management or prevention a PDE4 inhibitor. Without being limited by a particular theory, it is believed that a PDE4 inhibitor provided herein can promote bone formation by inhibiting osteoclastogenesis, inhibiting OCL activity, and/or inhibiting the production of SOST.

### 5.1 BRIEF DESCRIPTION OF FIGURES

FIG. 1 illustrates the effects of PDE4 inhibitor Apremilast® on osteoclastogenesis assessed through the number of TRAPS+ cells.

FIG. 2 also illustrates the effects of PDE4 inhibitor Apremilast® on osteoclastogenesis assessed through the number of TRAPS+ cells.

FIG. 3 illustrates the reduction of the levels of soluble RANKL in OCL cultures by PDE4 inhibitor Apremilast®.

FIG. 4 illustrates the inhibition of RANK gene expression in OCL cultures by PDE4 inhibitor Apremilast®.

FIG. 5 illustrates the increase of BMP-6 gene expression in OCL cultures by PDE4 inhibitor Apremilast®.

FIG. 6 illustrates the inhibition of OCL pit formation by PDE4 inhibitor Apremilast®.

FIG. 7 also illustrates the inhibition of OCL pit formation by PDE4 inhibitor Apremilast®.

FIG. 8 illustrates the reduction of soluble RANKL protein in OCL cultures by PDE4 inhibitor Apremilast®.

FIG. 9 illustrates the increase of OPG protein levels in OCL cultures by PDE4 inhibitor Apremilast®.

FIG. 10 shows the OBP cultures following treatment with PDE4 inhibitor Apremilast®.

FIG. 11 illustrates the effects of PDE4 inhibitor Apremilast® on soluble RANKL/OPG protein ratio.

FIG. 12A illustrates the schematics of OCY differentiation assay, wherein a 3D system for the culture of OCB with biphasic calcium phosphate particles is used to generate OCY.

FIG. 12B illustrates the successful differentiation of OCB into OCY using the 3D system.

FIG. 13 illustrates the effects of PDE4 inhibitor Apremilast® on expression of soluble RANKL protein in OCY.

FIG. 14 illustrates the effects of PDE4 inhibitor Apremilast® on expression of SOST protein in OCY.

### 5.2 PDE4 INHIBITORS

As used herein and unless otherwise indicated, the term “PDE4 inhibitor” encompasses small molecule drugs, e.g., small organic molecules which are not peptides, proteins, nucleic acids, oligosaccharides or other macromolecules. Preferred compounds inhibit TNF-α production. Further, the compounds may also have a modest inhibitory effect on LPS induced IL1β and IL12. More preferably, the compounds provided herein are potent PDE4 inhibitors.

PDE4 is one of the major phosphodiesterase isoenzymes found in human myeloid and lymphoid lineage cells. The enzyme plays a crucial part in regulating cellular activity by degrading the ubiquitous second messenger cAMP and maintaining it at low intracellular levels. Without being limited by theory, inhibition of PDE4 activity results in increased cAMP levels leading to the modulation of LPS induced cytokines, including inhibition of TNF-α production in monocytes as well as in lymphocytes.

In one embodiment, the PDE4 inhibitor is (+)-2-[1-(3-ethoxy-4-methoxyphenyl)-2-methanesulfonylethyl]-4-acetylaminoisoindolin-1,3-dione, Apremilast, which has the following structure:

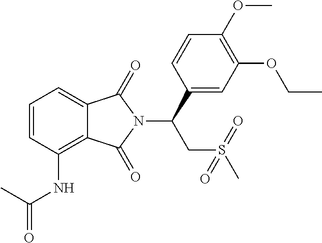 [SMILES: CCOc1cc([C@@H](CS(C)(=O)=O)N2C(=O)c3cccc(NC(C)=O)c3C2=O)ccc1OC]

or a pharmaceutically acceptable salt or solvate (e.g., hydrate) thereof.

Compounds provided herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of compounds provided herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds provided herein that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

2-[1-(3-ethoxy-4-methoxyphenyl)-2-methanesulfonylethyl]-4-acetylaminoisoindolin-1,3-dione contains one or chiral center, and can exist as a mixture of enantiomers. In one embodiment, provided herein is the use of stereomerically pure forms of 2-[1-(3-ethoxy-4-methoxyphenyl)-2-methanesulfonylethyl]-4-acetylaminoisoindolin-1,3-dione, as well as the use of mixtures of those forms. For example, mixtures comprising equal or unequal amounts of the enantiomers of 2-[1-(3-ethoxy-4-methoxyphenyl)-2-methanesulfonylethyl]-4-acetylaminoisoindolin-1,3-dione may be used in the methods and compositions provided herein. In one embodiment, the compound is stereomerically pure (+)-2-[1-(3-ethoxy-4-methoxyphenyl)-2-methanesulfonylethyl]-4-acetylaminoisoindolin-1,3-dione. The isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.3 SECOND ACTIVE AGENTS

(+)-2-[1-(3-ethoxy-4-methoxyphenyl)-2-methanesulfonylethyl]-4-acetylaminoisoindolin-1,3-dione (“Apremilast®”) may be combined with other pharmacologically active compounds (“second active agents”) in the methods and compositions provided herein. It is believed that certain combinations work synergistically in the treatment, prevention and/or management of bone loss. (+)-2-[1-(3-ethoxy-4-methoxyphenyl)-2-methanesulfonylethyl]-4-acetylaminoisoindolin-1,3-dione may also work to alleviate adverse effects associated with certain second active agents, and some second active agents can be used to alleviate adverse effects associated with the administration of (+)-2-[1-(3-ethoxy-4-methoxyphenyl)-2-methanesulfonylethyl]-4-acetylaminoisoindolin-1,3-dione.

In some embodiments, second agents are medications and therapy conventionally used to treat bone loss, e.g., osteoporosis. Examples of second active agents include, but are not limited to: a bisphosphonate, teriparatide, strontium renelate, raloxifene, denosumab, calcium, vitamin D and vitamin K.

### 5.4 METHODS OF TREATMENTS AND PREVENTION

Provided herein are methods of treating, managing or preventing loss of bone comprising administering to a patient in need of such treatment, management or prevention a PDE4 inhibitor. In one embodiment, the bone loss is a primary bone loss, e.g., primary type osteoporosis. In another embodiment, the bone loss is secondary to other underlying disorders, e.g., secondary type osteoporosis. In another embodiment, the bone loss is caused by therapeutic agents that are used to treat other disorders.

In one embodiment, the loss of bone is primary type 1 osteoporosis, e.g., those that occur in women after the menopause. In another embodiment, the loss of bone is primary type 2 osteoporosis, e.g., those that occur in senile patients who are typically 75 years of age or older.

In one embodiment, the loss of bone is secondary to other underlying disorders. Examples of underlying disorders include, but are not limited to: an autoimmune disorder such as rheumatoid arthritis, lupus, multiple sclerosis and ankylosing spondylitis; a digestive or gastrointestinal disorder such as celiac disease and inflammatory bowel disease; an endocrine/hormonal disorder such as diabetes, hyperparathyroidism, hyperthyroidism, Cushing's syndrome, thyrotoxicosis, premature menopause, and unusual testosterone levels; a hematologic disorder such as leukemia, lymphoma, multiple myeloma, sickle cell disease, a blood and bone marrow disorder; and thalassemia; a neurological disorder such as stroke, Parkinson's disease, multiple sclerosis and spinal cord injury; a mental illness such as depression and an eating disorder (e.g., anorexia nervosa); cancer such as bone, breast and prostate cancer; and other disorders such as AIDS, female athlete triad, a kidney disease, a liver disease, polio, post-polio syndrome, malnutrition, scoliosis, and unusual weight loss.

In one embodiment, the underlying disorder is rheumatoid arthritis. In another embodiment, the underlying disorder is multiple sclerosis. In another embodiment, the underlying disorder is multiple myeloma.

In certain embodiment, also provided herein is a method of treating, preventing and/or managing loss of bone caused by medications or therapies used for treatment of other disorders. Such medications or therapies include, but are not limited to: steroid (e.g., corticosteroid) such as prednisone and dexamethasone; an antiepileptic such as a barbiturate and phenytoin; L-thyroxine; an aromatase inhibitor; methotrexate; depot-progesterone; a gonadotropin-releasing hormone agonist; a proton pump inhibitor; a thiazolidinedione; lithium; gastric bypass surgery; and organ transplant. In one embodiment, the medication is corticosteroid. In another embodiment, the medication is dexamethasone. In another embodiment, the medication is prednisone.

In certain embodiments, also provided herein is a method of treating, preventing and/or managing the variations or symptoms of osteoporosis. Examples include, but are not limited to, osteogenesis imperfecta, osteomalacia, rickets, ostesis fibrosa cystic and Paget's disease.

In one embodiment, the PDE4 inhibitor is (+)-2-[1-(3-ethoxy-4-methoxyphenyl)-2-methanesulfonylethyl]-4-acetylaminoisoindolin-1,3-dione, which has the following chemical structure:

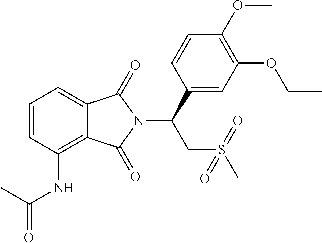 [SMILES: CCOc1cc([C@@H](CS(C)(=O)=O)N2C(=O)c3cccc(NC(C)=O)c3C2=O)ccc1OC]

or a pharmaceutically acceptable salt or solvate (e.g., hydrate) thereof.

In one embodiment, provided herein is a method of treating, preventing and/or managing bone loss, which comprises administering to a patient a therapeutically or prophylactically effective amount of (+)-2-[1-(3-ethoxy-4-methoxyphenyl)-2-methanesulfonylethyl]-4-acetylaminoisoindolin-1,3-dione, or a pharmaceutically acceptable salt or solvate thereof.

In one embodiment, the compound is administered in an amount of from about 1 to about 100 mg per day.

In one embodiment, the compound is administered in an amount of about 20, 40, 60, 80 or 100 mg per day.

In one embodiment, the compound is administered in an amount of about 20 mg, twice per day.

In one embodiment, the compound is administered in an amount of about 30 mg, twice per day.

In one embodiment, the compound is orally administered.

In one embodiment, the compound is administered in a capsule or tablet.

Also provided herein are pharmaceutical compositions (e.g., single unit dosage forms) that can be used in methods disclosed herein. Particular pharmaceutical compositions comprise a compound as provided herein, or a pharmaceutically acceptable salt, solvate, hydrate, clathrate, or prodrug thereof, and a second active agent.

As used herein, unless otherwise specified, the term “treating” refers to the administration of a compound provided herein or other additional active agent after the onset of symptoms of the bone loss. As used herein, unless otherwise specified, the term “preventing” refers to the administration prior to the onset of symptoms, particularly to patients at risk of bone loss. The term “prevention” includes the inhibition of a symptom of bone loss. As used herein and unless otherwise indicated, the term “managing” encompasses preventing the recurrence of bone loss in a patient who had suffered from it, and/or lengthening the time a patient who had suffered from bone loss remains in remission.

As used herein, and unless otherwise specified, the terms “bone loss” and “loss of bone” encompass all abnormalities in mass, strength and structures of the bones. Examples include, but are not limited to, decreased bone mass, change in bone density, bone softness, tumors on bones and abnormal bone architecture.

The term “therapeutically effective amount” refers to an amount of a compound or composition that, when administered to a subject for treating bone loss, is sufficient to effect such treatment for the disorder. A “therapeutically effective amount” can vary depending on, inter alia, the compound, the disease and its severity, and the age, weight, etc., of the subject to be treated.

Provided herein are methods of treating patients who have been previously treated but are non-responsive to standard therapies, as well as those who have not previously been treated. Also provided herein are methods of treating patients regardless of patient's age, although some diseases or disorders are more common in certain age groups. Also provided herein are methods of treating patients who have undergone surgery in an attempt to treat the disease or condition at issue, as well as those who have not.

Because patients with bone loss have heterogeneous clinical manifestations and varying clinical outcomes, the treatment given to a patient may vary, depending on his/her prognosis. The skilled clinician will be able to readily determine without undue experimentation specific secondary agents, types of therapy that can be effectively used to treat an individual patient with bone loss.

In some embodiments, (+)-2-[1-(3-ethoxy-4-methoxyphenyl)-2-methanesulfonylethyl]-4-acetylaminoisoindolin-1,3-dione can be administered orally and in single or divided daily doses in an amount of from about 0.10 to about 150 mg/day. In one embodiment, (+)-2-[1-(3-ethoxy-4-methoxyphenyl)-2-methanesulfonylethyl]-4-acetylaminoisoindolin-1,3-dione may be administered in an amount of from about 10 to about 50 mg per day, about 5 to 25 mg per day, or alternatively from about 10 to about 50 mg every other day.

In one embodiment, (+)-2-[1-(3-ethoxy-4-methoxyphenyl)-2-methanesulfonylethyl]-4-acetylaminoisoindolin-1,3-dione may be administered in an amount of about 1, 2.5, 3, 4, 5, 6, 7, 8, 9, 10, 15, 20, 25, 30, 35, 40, 45, 50, 60, 70, 80, 90 or 100 mg per day. In another embodiment, (+)-2-[1-(3-ethoxy-4-methoxyphenyl)-2-methanesulfonylethyl]-4-acetylaminoisoindolin-1,3-dione may be administered in an amount of about 20, 40, 60, 80 or 100 mg per day. In yet another embodiment, (+)-2-[1-(3-ethoxy-4-methoxyphenyl)-2-methanesulfonylethyl]-4-acetylaminoisoindolin-1,3-dione may be administered in an amount of about 10, 20, 25, 40 or 50 mg per day. In another embodiment, (+)-2-[1-(3-ethoxy-4-methoxyphenyl)-2-methanesulfonylethyl]-4-acetylaminoisoindolin-1,3-dione may be administered initially in an amount of 5 mg/day and the dose can be escalated every week to 10, 20, 25, 30, 40 and 50 mg/day. In another embodiment, (+)-2-[1-(3-ethoxy-4-methoxyphenyl)-2-methanesulfonylethyl]-4-acetylaminoisoindolin-1,3-dione may be administered in an amount of 20 mg twice per day.

### 5.4.1 Combination Therapy with a Second Active Agent

Specific methods provided herein comprise administering (+)-2-[1-(3-ethoxy-4-methoxyphenyl)-2-methanesulfonylethyl]-4-acetylaminoisoindolin-1,3-dione, or a pharmaceutically acceptable salt, solvate, hydrate, clathrate, or prodrug thereof, in combination with one or more second active agents. Examples of second active agents are also disclosed herein (see, e.g., section 4.3).

In one embodiment, the additional active agent is a bisphosphonate, teriparatide, strontium renelate, raloxifene, denosumab, calcium, vitamin D, vitamin K or a combination thereof.

In one embodiment, the additional active agent is a biphosphonate.

In another embodiment, the additional active agent is calcium.

In another embodiment, the additional active agent is vitamin D.

Administration of (+)-2-[1-(3-ethoxy-4-methoxyphenyl)-2-methanesulfonylethyl]-4-acetylaminoisoindolin-1,3-dione and second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration (+)-2-[1-(3-ethoxy-4-methoxyphenyl)-2-methanesulfonylethyl]-4-acetylaminoisoindolin-1,3-dione is oral. Preferred routes of administration for the second active agents are known to those of ordinary skill in the art. See, e.g., Physicians' Desk Reference, 1755-1760 (56^{th} ed., 2002).

In one embodiment, the second active agent is administered orally, intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the severity and stage of disease, the amount(s) of the first compound, and any optional additional active agents concurrently administered to the patient. In a particular embodiment, the second active agent is a corticosteroid (e.g., prednisone), methotrexate, azathioprine, hydroxychloroquine, cyclophosphamide, minocycline, doxycycline, chloroquin, infliximab, a penicillin antibiotic, a cephalosporin antibiotic, a macrolide antibiotic, a lincomycin antibiotic, a tetracycline antibiotic, or a combination thereof.

### 5.4.2 Cycling Therapy

In certain embodiments, the prophylactic or therapeutic agents provided herein are cyclically administered to a patient. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one embodiment, (+)-2-[1-(3-ethoxy-4-methoxyphenyl)-2-methanesulfonylethyl]-4-acetylaminoisoindolin-1,3-dione, or a pharmaceutically acceptable salt, solvate, hydrate, clathrate, or prodrug thereof, is administered daily in a single or divided doses in a four to six week cycle with a rest period of about a week or two weeks. In some embodiments, the frequency, number, and length of dosing cycles may be increased. Thus, another embodiment encompasses the administration of (+)-2-[1-(3-ethoxy-4-methoxyphenyl)-2-methanesulfonylethyl]-4-acetylaminoisoindolin-1,3-dione for more cycles than are typical when it is administered alone. In yet another embodiment, (+)-2-[1-(3-ethoxy-4-methoxyphenyl)-2-methanesulfonylethyl]-4-acetylaminoisoindolin-1,3-dione is administered for a greater number of cycles that would typically cause dose-limiting toxicity in a patient to whom a second active ingredient is not also being administered.

In one embodiment, (+)-2-[1-(3-ethoxy-4-methoxyphenyl)-2-methanesulfonylethyl]-4-acetylaminoisoindolin-1,3-dione, or a pharmaceutically acceptable salt, solvate, hydrate, clathrate, or prodrug thereof, is administered daily and continuously for three or four weeks at a dose of from about 0.1 to about 150 mg/d followed by a break of one or two weeks. In one embodiment, (+)-2-[1-(3-ethoxy-4-methoxyphenyl)-2-methanesulfonylethyl]-4-acetylaminoisoindolin-1,3-dione, or a pharmaceutically acceptable salt, solvate, hydrate, clathrate, or prodrug thereof, is administered in an amount of about 20 mg twice per day for three to four weeks, followed by one week or two weeks of rest in a four or six week cycle.

In one embodiment, (+)-2-[1-(3-ethoxy-4-methoxyphenyl)-2-methanesulfonylethyl]-4-acetylaminoisoindolin-1,3-dione, or a pharmaceutically acceptable salt, solvate, hydrate, clathrate, or prodrug thereof, and a second active agent are administered orally, with administration of (+)-2-[1-(3-ethoxy-4-methoxyphenyl)-2-methanesulfonylethyl]-4-acetylaminoisoindolin-1,3-dione occurring 30 to 60 minutes prior to a second active agent, during a cycle of four to six weeks. Typically, the number of cycles during which the combinatorial treatment is administered to a patient will be from about one to about 24 cycles, more typically from about two to about 16 cycles, and even more typically from about four to about three cycles.

### 5.5 PHARMACEUTICAL COMPOSITIONS AND DOSAGE FORMS

Pharmaceutical compositions can be used in the preparation of individual, single unit dosage forms. Pharmaceutical compositions and dosage forms provided herein comprise (+)-2-[1-(3-ethoxy-4-methoxyphenyl)-2-methanesulfonylethyl]-4-acetylaminoisoindolin-1,3-dione, or a pharmaceutically acceptable salt, solvate, hydrate, clathrate, or prodrug thereof. Pharmaceutical compositions and dosage forms may further comprise one or more excipients.

Pharmaceutical compositions and dosage forms may also comprise one or more additional active ingredients. Consequently, pharmaceutical compositions and dosage forms provided herein comprise the active agents disclosed herein (e.g., (+)-2-[1-(3-ethoxy-4-methoxyphenyl)-2-methanesulfonylethyl]-4-acetylaminoisoindolin-1,3-dione and a second active agent). Examples of optional second, or additional, active agents are disclosed herein (see, e.g., section 4.2).

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage form will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms provided herein will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions may comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

Also provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms may be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms provided herein comprise (+)-2-[1-(3-ethoxy-4-methoxyphenyl)-2-methanesulfonylethyl]-4-acetylaminoisoindolin-1,3-dione or a pharmaceutically acceptable salt, solvate, hydrate, clathrate, or prodrug thereof in an amount of from about 0.10 to about 150 mg. Typical dosage forms comprise (+)-2-[1-(3-ethoxy-4-methoxyphenyl)-2-methanesulfonylethyl]-4-acetylaminoisoindolin-1,3-dione or a pharmaceutically acceptable salt, solvate, hydrate, clathrate, or prodrug thereof in an amount of about 0.1, 1, 2, 5, 7.5, 10, 12.5, 15, 17.5, 20, 25, 50, 100, 150 or 200 mg. In a specific embodiment, a preferred dosage form comprises (+)-2-[1-(3-ethoxy-4-methoxyphenyl)-2-methanesulfonylethyl]-4-acetylaminoisoindolin-1,3-dione in an amount of about 5, 10, 20, 25 or 50 mg. Typical dosage forms comprise the second active agent in an amount of 1 to about 1000 mg, from about 5 to about 500 mg, from about 10 to about 350 mg, or from about 50 to about 200 mg. Of course, the specific amount of second active agent will depend on the specific agent used, the amounts of (+)-2-[1-(3-ethoxy-4-methoxyphenyl)-2-methanesulfonylethyl]-4-acetylaminoisoindolin-1,3-dione and any optional additional active agents concurrently administered to the patient.

### 5.5.1 Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent. Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof.

An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is typically present in from about 50 to about 99 weight percent of the pharmaceutical composition or dosage form.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof.

Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A specific solid oral dosage form provided herein comprises (+)-2-[1-(3-ethoxy-4-methoxyphenyl)-2-methanesulfonylethyl]-4-acetylaminoisoindolin-1,3-dione, or a pharmaceutically acceptable salt or solvate thereof, lactose anhydrous, microcrystalline cellulose, croscarmellose sodium and magnesium stearate.

### 5.5.2 Delayed Release Dosage Forms

Active ingredients provided herein may be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein. Thus, provided herein are single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 5.5.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms provided herein. For example, cyclodextrin and its derivatives can be used to increase the solubility of a compound provided herein and its derivatives. See, e.g., U.S. Pat. No. 5,134,127, which is incorporated herein by reference.

### 5.5.4 Topical and Mucosal Dosage Forms

Topical and mucosal dosage forms include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms provided herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.5.5 Kits

Typically, active ingredients provided herein are preferably not administered to a patient at the same time or by the same route of administration. Therefore, provided herein are kits which, when used by the medical practitioner, can simplify the administration of appropriate amounts of active ingredients to a patient.

A typical kit provided herein comprises a dosage form of (+)-2-[1-(3-ethoxy-4-methoxyphenyl)-2-methanesulfonylethyl]-4-acetylaminoisoindolin-1,3-dione, or a pharmaceutically acceptable salt, solvate, hydrate, prodrug, or clathrate thereof. Kits provided herein may further comprise additional active ingredients such as a corticosteroid (e.g., prednisone), methotrexate, azathioprine, hydroxychloroquine, cyclophosphamide, minocycline, doxycycline, chloroquin, infliximab, a penicillin antibiotic, a cephalosporin antibiotic, a macrolide antibiotic, a lincomycin antibiotic, a tetracycline antibiotic, or a combination thereof. Examples of the additional active ingredients include, but are not limited to, those disclosed herein (see, e.g., section 4.2). Kits provided herein may further comprise devices that are used to administer the active ingredients. Examples of such devices include, but are not limited to, syringes, drip bags, patches, and inhalers.

Kits may further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to:

Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6. EXAMPLES

Certain embodiments provided herein are illustrated by the following non-limiting examples.

### 6.1 PREPARATION OF (+)-2-[1-(3-ETHOXY-4-METHOXYPHENYL)-2-METHANESULFONYLETHYL]-4-ACETYLAMINOISOINDOLIN-1,3-DIONE (APREMILAST)

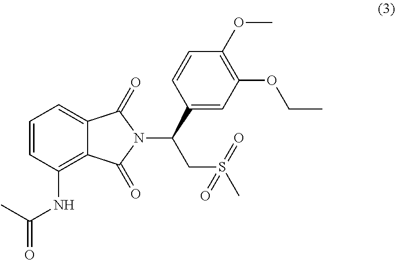 [SMILES: CCOc1cc([C@@H](CS(C)(=O)=O)N2C(=O)c3cccc(NC(C)=O)c3C2=O)ccc1OC]

### 6.1.1 Preparation of 3-aminopthalic acid

10% Pd/C (2.5 g), 3-nitrophthalic acid (75.0 g, 355 mmol) and ethanol (1.5 L) were charged to a 2.5 L Parr hydrogenator under a nitrogen atmosphere. Hydrogen was charged to the reaction vessel for up to 55 psi. The mixture was shaken for 13 hours, maintaining hydrogen pressure between 50 and 55 psi. Hydrogen was released and the mixture was purged with nitrogen 3 times. The suspension was filtered through a celite bed and rinsed with methanol. The filtrate was concentrated in vacuo. The resulting solid was reslurried in ether and isolated by vacuum filtration. The solid was dried in vacua to a constant weight, affording 54 g (84% yield) of 3-aminopthalic acid as a yellow product. ¹H-NMR (DMSO-d₆) δ: 3.17 (s, 2H), 6.67 (d, 1H), 6.82 (d, 1H), 7.17 (t, 1H), 8-10 (brs, 2H). ¹³C-NMR (DMSO-d₆) δ: 112.00, 115.32, 118.20, 131.28, 135.86, 148.82, 169.15, 170.09.

### 6.1.2 Preparation of 3-acetamidopthalic anhydride

A 1 L 3-necked round bottom flask was equipped with a mechanical stirrer, thermometer, and condenser and charged with 3-aminophthalic acid (108 g, 596 mmol) and acetic anhydride (550 mL). The reaction mixture was heated to reflux for 3 hours and cooled to ambient temperature and further to 0-5.degree. C. for another 1 hour. The crystalline solid was collected by vacuum filtration and washed with ether. The solid product was dried in vacua at ambient temperature to a constant weight, giving 75 g (61% yield) of 3-acetamidopthalic anhydride as a white product. ¹H-NMR (CDCl₃) δ: 2.21 (s, 3H), 7.76 (d, 1H), 7.94 (t, 1H), 8.42 (d, 1H), 9.84 (s, 1H).

### 6.1.3 Resolution of 2-(3-ethoxy-4-methoxyphenyl)-1-(methylsulphonyl)-ethyl-2-amine

A 3 L 3-necked round bottom flask was equipped with a mechanical stirrer, thermometer, and condenser and charged with 2-(3-ethoxy-4-methoxyphenyl)-1-(methylsulphonyl)-eth-2-ylamine (137.0 g, 500 mmol), N-acetyl-L-leucine (52 g, 300 mmol), and methanol (1.0 L). The stirred slurry was heated to reflux for 1 hour. The stirred mixture was allowed to cool to ambient temperature and stirring was continued for another 3 hours at ambient temperature. The slurry was filtered and washed with methanol (250 mL). The solid was air-dried and then dried in vacuo at ambient temperature to a constant weight, giving 109.5 g (98% yield) of the crude product (85.8% ee). The crude solid (55.0 g) and methanol (440 mL) were brought to reflux for 1 hour, cooled to room temperature and stirred for an additional 3 hours at ambient temperature. The slurry was filtered and the filter cake was washed with methanol (200 mL). The solid was air-dried and then dried in vacuo at 30° C. to a constant weight, yielding 49.6 g (90% recovery) of (S)-2-(3-ethoxy-4-methoxyphenyl)-1-(methylsulphonyl)-eth-2-ylamine-N-acetyl-L-leucine salt (98.4% ee). Chiral HPLC (1/99 EtOH/20 mM KH₂PO₄ @pH 7.0, Ultron Chiral ES-OVS from Agilent Technologies, 150 mm.times.4.6 mm, 0.5 mL/min., @240 nm): 18.4 min (S-isomer, 99.2%), 25.5 min (R-isomer, 0.8%)

### 6.1.4 Preparation of (+)-2-[1-(3-ethoxy-4-methoxyphenyl)-2-methanesulfonylethyl]-4-acetylaminoisoindolin-1,3-dione

A 500 mL 3-necked round bottom flask was equipped with a mechanical stirrer, thermometer, and condenser. The reaction vessel was charged with (S)-2-(3-ethoxy-4-methoxyphenyl)-1-(methylsulphonyl)-eth-2-yl amine N-acetyl-L-leucine salt (25 g, 56 mmol, 98% ee), 3-acetamidophthalic anhydride (12.1 g, 58.8 mmol), and glacial acetic acid (250 mL). The mixture was refluxed over night and then cooled to <50° C. The solvent was removed in vacuo, and the residue was dissolved in ethyl acetate. The resulting solution was washed with water (250 mL×2), saturated aqeous NaHCO₃ (250 mL.times.2), brine (250 mL.times.2), and dried over sodium sulphate. The solvent was evaporated in vacuo, and the residue recrystallized from a binary solvent containing ethanol (150 mL) and acetone (75 mL). The solid was isolated by vacuum filtration and washed with ethanol (100 mL.times.2). The product was dried in vacuo at 60° C. to a constant weight, affording 19.4 g (75% yield) of Compound 3 with 98% ee. Chiral HPLC (15/85 EtOH/20 mM KH₂PO₄ @pH 3.5, Ultron Chiral ES-OVS from Agilent Technology, 150 mm×4.6 mm, 0.4 mL/min., @240 nm): 25.4 min (S-isomer, 98.7%), 29.5 min (R-isomer, 1.2%). ¹H-NMR (CDCl₃) δ: 1.47 (t, 3H), 2.26 (s, 3H), 2.87 (s, 3H), 3.68-3.75 (dd, 1H), 3.85 (s, 3H), 4.07-4.15 (q, 2H), 4.51-4.61 (dd, 1H), 5.84-5.90 (dd, 1H), 6.82-8.77 (m, 6H), 9.46 (s, 1H). ¹³C-NMR (DMSO-d₆) δ: 14.66, 24.92, 41.61, 48.53, 54.46, 55.91, 64.51, 111.44, 112.40, 115.10, 118.20, 120.28, 124.94, 129.22, 131.02, 136.09, 137.60, 148.62, 149.74, 167.46, 169.14, 169.48.

### 6.2 INHIBITION OF PDE4

Phosphodiesterase 4 enzyme was purified from U937 human monocytic cells by gel filtration chromatography, and phosphodiesterase reactions were carried out as previously described. See, e.g., Muller et al., Bioorg. Med. Chem. Lett., 1998, 8(19): 2669-2674. Briefly, reactions were carried out in 96-well deep-well plates in 50 mM Tris HCl pH 7.5, 5 mM MgCl₂, 1 μM cyclic adenosine monophosphate (cAMP), plus 10 nM [³H]-cAMP for 45 min at 30° C. The reactions were terminated by boiling, treated with 1 mg/ml snake venom, and separated using AG-1X8 ion exchange resin (BioRad). Reactions consumed less than 15% of available substrate. (+)-2-[1-(3-ethoxy-4-methoxyphenyl)-2-methanesulfonylethyl]-4-acetylaminoisoindolin-1,3-dione inhibited PDE4 with an IC₅₀ of 73.5 nM.

### 6.3 EFFECTS ON OCL, OBL AND OCY

### 5.3.1 Procedures

OCL Differentiation and Pit Formation

Human bone marrow mononuclear cells were differentiated into OCL using 10 nM dexamethasone and 10 nM vitamin D for 7 days. APR (0.1-10 μM) was added with fresh medium on days 0 and 3. The following compounds were also used: the prototypic PDE4 inhibitor rolipram (ROL) at 10 μM, the bisphosphonate alendronate (ALEN) at 10 μM, and sulfasalazine (SULF) at 30 μM. OCL were stained for tartrate-resistant acid phosphatase 5 (TRAPS), and OBL were stained for alkaline phosphatase (ALP). OCL were lysed and RNA was isolated and converted to cDNA. Reverse transcription polymerase chain reaction (RT-PCR) was performed to measure gene expression of RANK and bone morphogenetic protein 6 (BMP-6). For OCL pit formation, RAW 264.7 mouse macrophages were plated in Corning 24-well Osteo Surface plates and incubated for 2 hours to allow attachment. RANKL (50 ng/mL) was added to each well along with compounds and medium on days 0 and 3. Cells were removed with bleach and pictures of the wells were taken to assess OCL activity. NIH ImageJ software was used for image analysis.

OBL Culture

Normal human OBL (nHOST, Lonza) were grown using OBL basal medium containing 10% fetal bovine serum, ascorbic acid, and gentamicin/amphotericin-B (Lonza). Cells were grown to a density of 5×10⁵ cells/mL. Compounds were added and the final concentration of dimethyl sulfoxide (DMSO) was 0.25%. Plates were incubated at 5% CO₂, 37° C. for 7 days, changing media every 3 days and adding fresh compound. The supernatant was collected for analysis of RANKL (USCNK Life Science Inc), OPG (R&D Systems), and SOST (R&D Systems) by enzyme-linked immunosorbent assay (ELISA). Cells were stained for ALP.

OCY Differentiation

OBL were differentiated into OCY using hydroxyapatite/tricalcium phosphate biphasic calcium phosphate ceramic particles (Graftys BCP), which were placed into polycarbonate filter well inserts, and the cell culture media was changed every 3 days for a total of 28 days. Gene expression was measured by quantitative RT-PCR for the following: RANK, RANKL, SOST, OPG, mothers against decapentaplegic homolog 1 (SMAD1), collagen 9A2 (COL9A2), vascular endothelial growth factor C (VEGFC), and fibroblast growth factor receptor 1 (FGFR1). Protein production was measured by ELISA.

### 5.3.2 Results

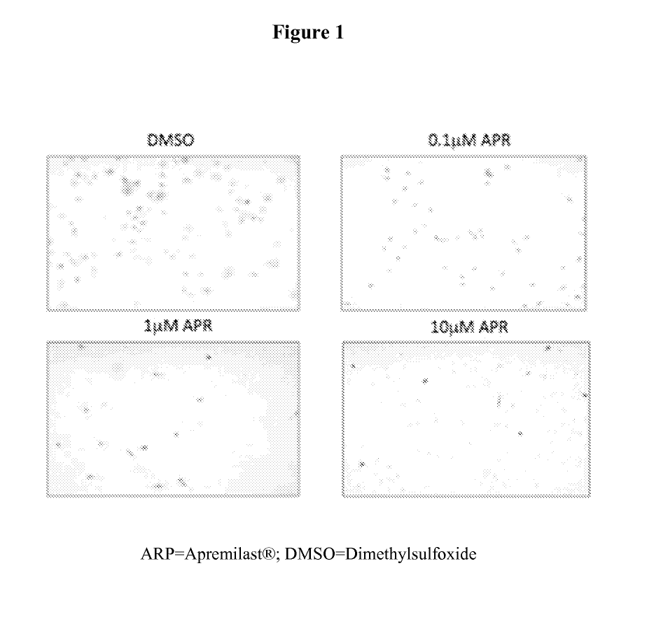

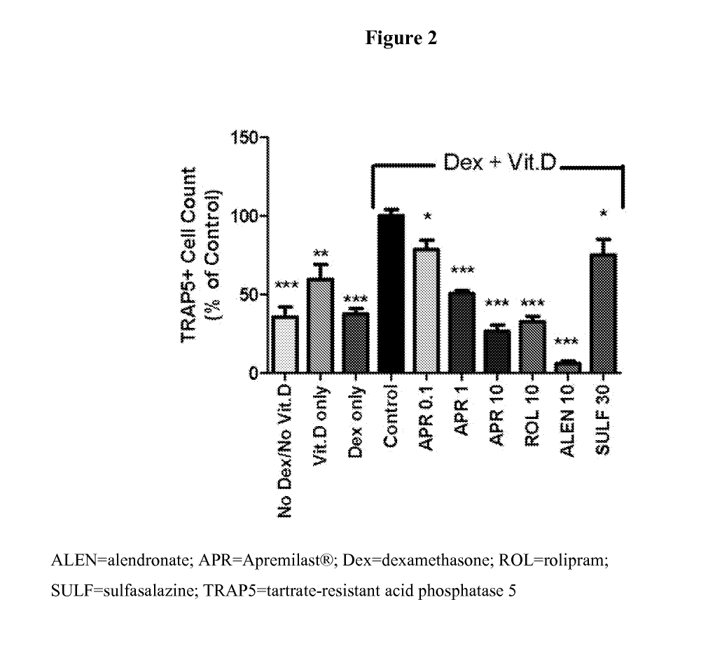

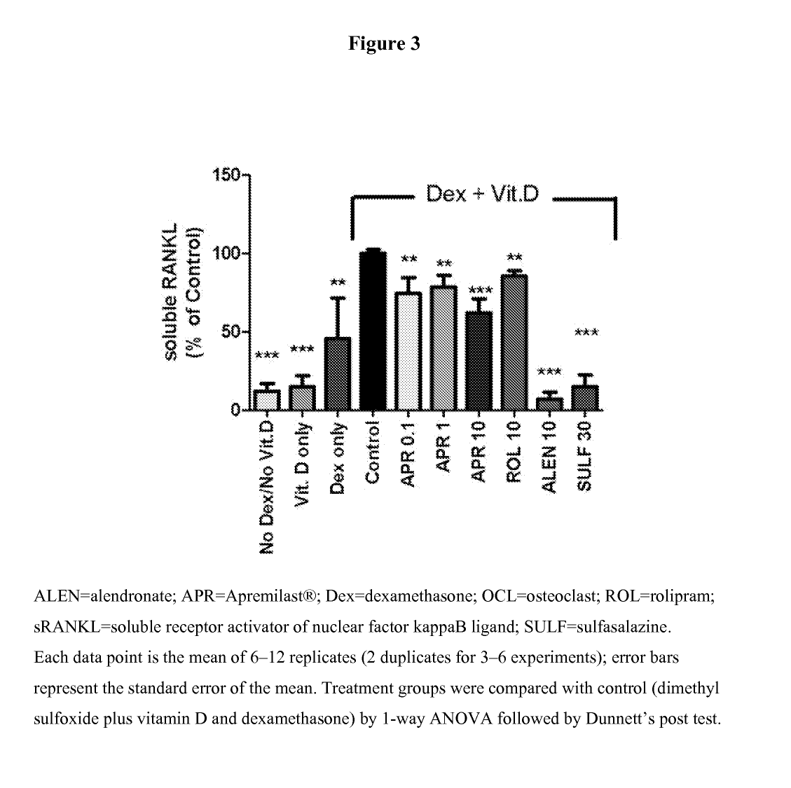

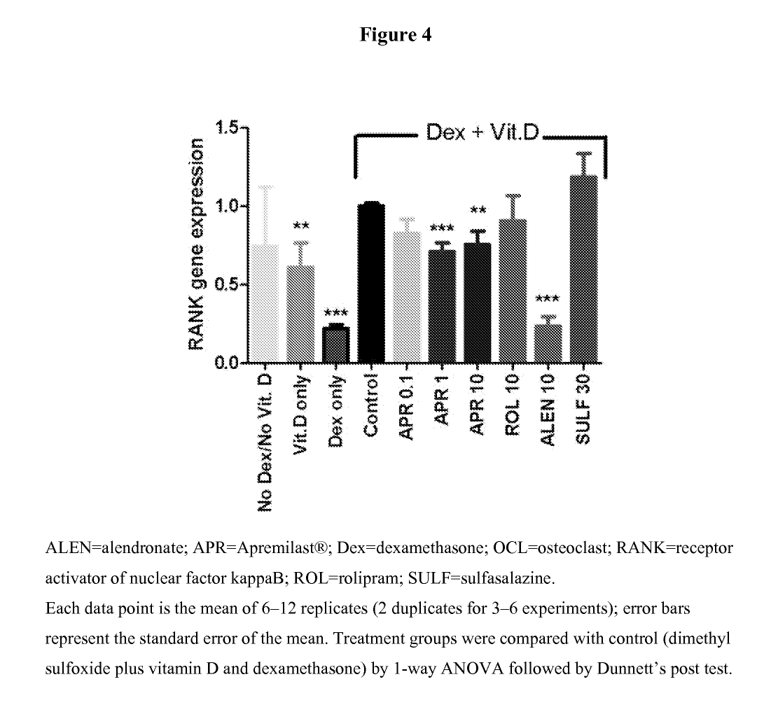

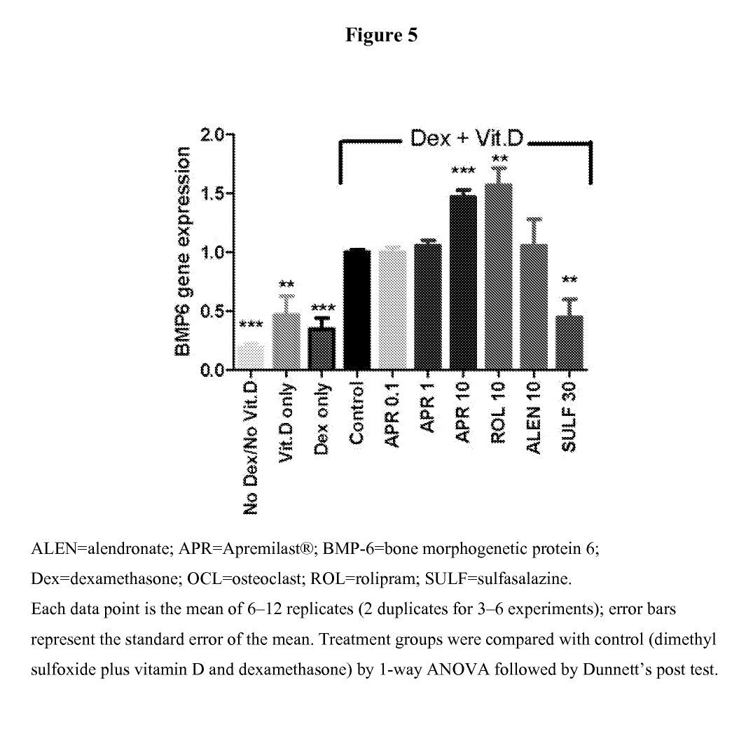

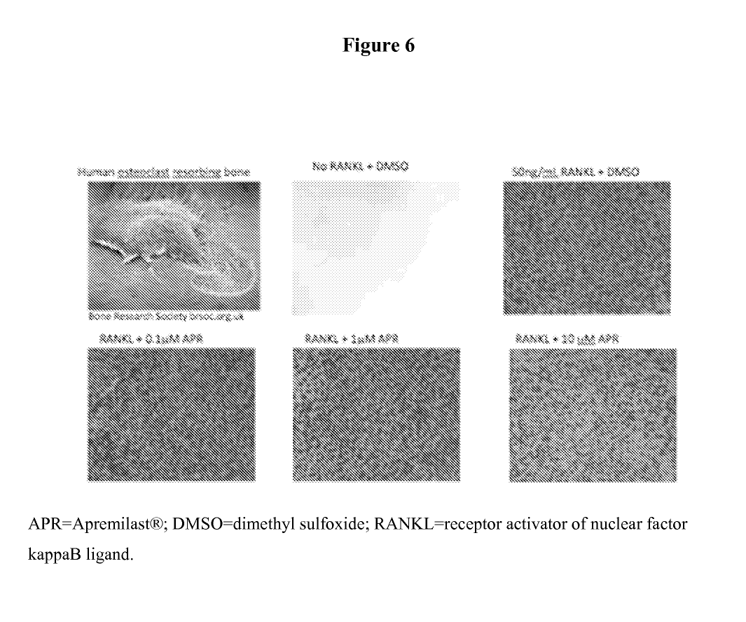

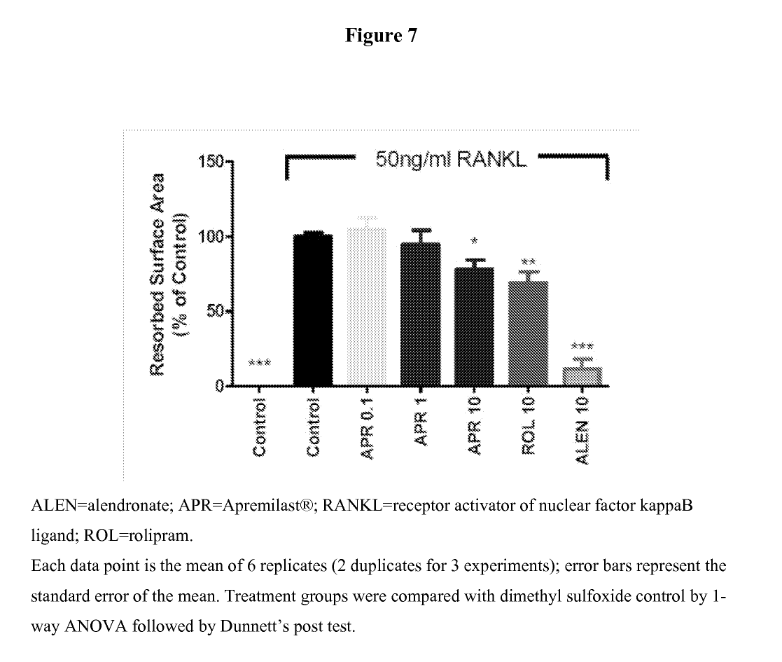

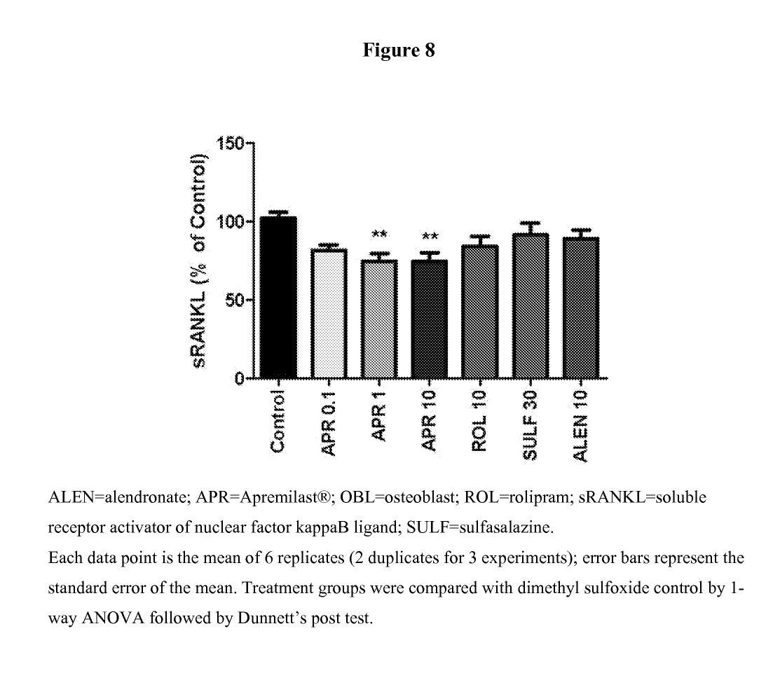

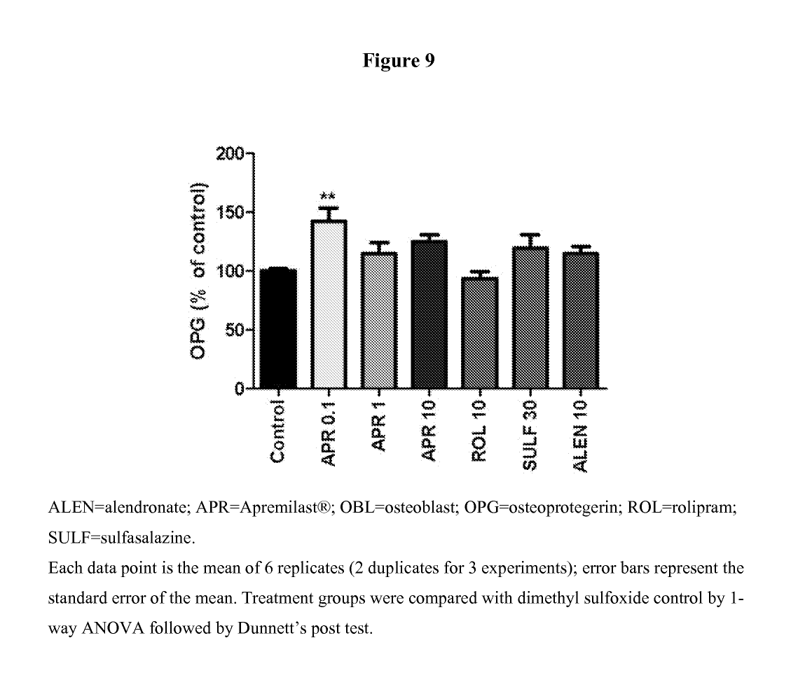

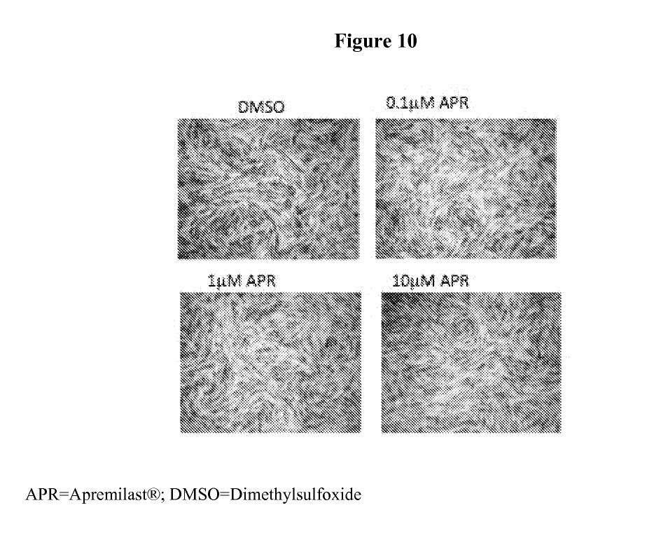

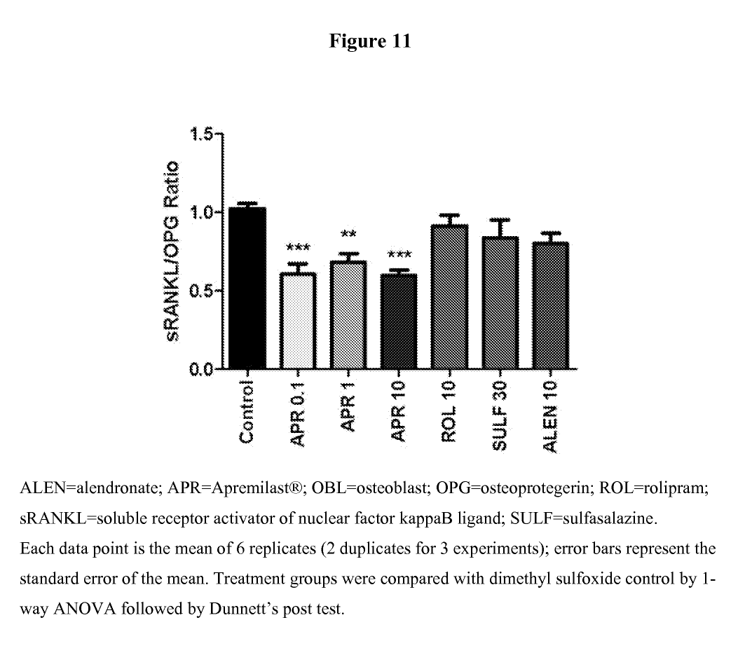

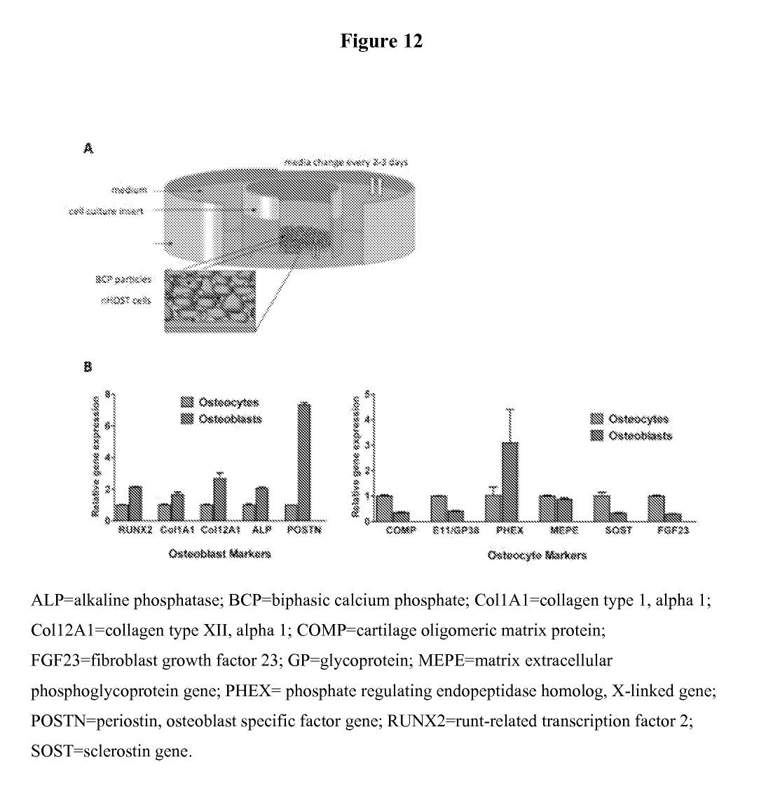

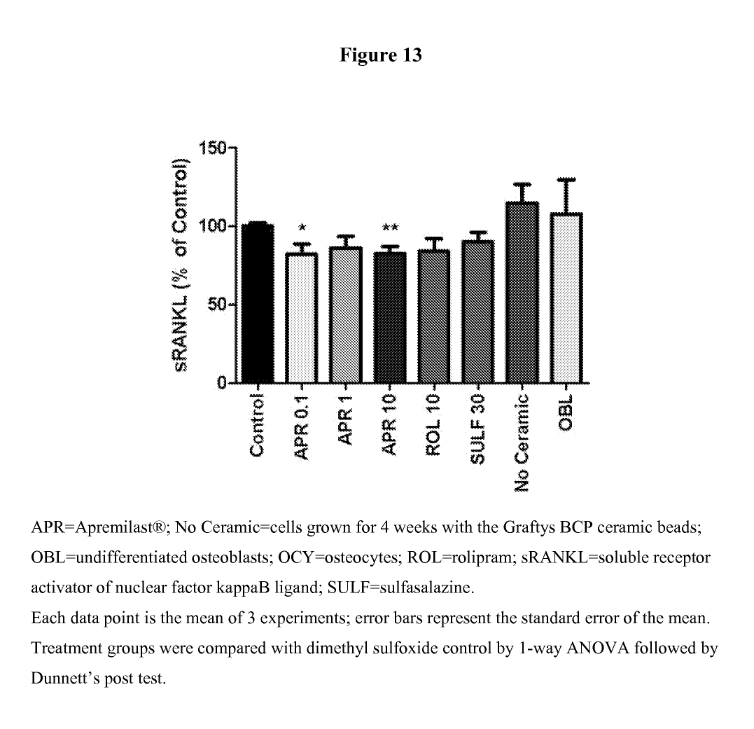

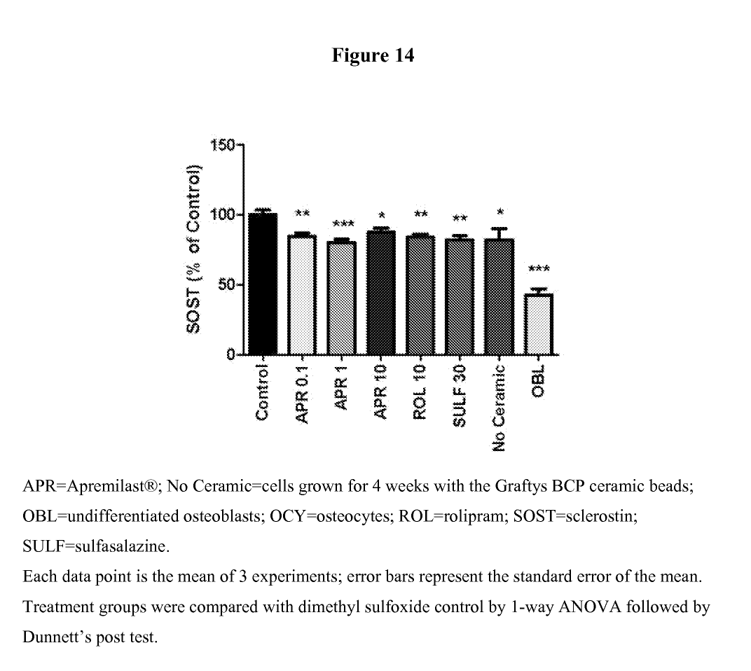

In OCL cultures, the number of TRAP-5+ cells was reduced by (+)-2-[1-(3-ethoxy-4-methoxyphenyl)-2-methanesulfonylethyl]-4-acetylaminoisoindolin-1,3-dione by 21%, 49%, and 73% at 0.1 μM, 1 μM, and 10 μM, respectively. In the OCL cultures, (+)-2-[1-(3-ethoxy-4-methoxyphenyl)-2-methanesulfonylethyl]-4-acetylaminoisoindolin-1,3-dione significantly reduced the levels of sRANKL protein by 25%, 21%, and 38% at 0.1 μM, 1 μM, and 10 μM, respectively. (+)-2-[1-(3-ethoxy-4-methoxyphenyl)-2-methanesulfonylethyl]-4-acetylaminoisoindolin-1,3-dione at 1 μM and 10 μM significantly inhibited RANK gene expression by 30% and 25%, respectively. Alendronate inhibited RANK gene expression by 77%. In OBL, (+)-2-[1-(3-ethoxy-4-methoxyphenyl)-2-methanesulfonylethyl]-4-acetylaminoisoindolin-1,3-dione reduced sRANKL protein levels by 25% at both 1 μM and 10 μM. Rolipram, alendronate, and sulfasalazine all had no significant effect on sRANKL protein levels in the OBL supernatants. In addition, (+)-2-[1-(3-ethoxy-4-methoxyphenyl)-2-methanesulfonylethyl]-4-acetylaminoisoindolin-1,3-dione significantly increased OPG protein levels by 42% at 0.1 μM. Overall, (+)-2-[1-(3-ethoxy-4-methoxyphenyl)-2-methanesulfonylethyl]-4-acetylaminoisoindolin-1,3-dione decreased the sRANKL/OPG protein ratio by 39%, 32%, and 40% at 0.1 μM, 1 μM, and 10 μM, respectively. By comparison, rolipram, alendronate, and sulfasalazine had no effect on the sRANKL/OPG ratio. In OCY, (+)-2-[1-(3-ethoxy-4-methoxyphenyl)-2-methanesulfonylethyl]-4-acetylaminoisoindolin-1,3-dione significantly reduced sRANKL production by 18%, 14%, and 17% at 0.1 μM, 1 μM, and 10 μM. APR also significantly reduced SOST protein levels by 16%, 20%, and 14% at 0.1 μM, 1 μM, and 10 μM. These results were shown in FIGS. 1-14.

These results demonstrate that (+)-2-[1-(3-ethoxy-4-methoxyphenyl)-2-methanesulfonylethyl]-4-acetylaminoisoindolin-1,3-dione inhibits osteoclastogenesis in vitro at clinically relevant concentrations (0.1-1 μM). This effect was associated with a decrease in sRANKL protein expression by OBL, but also may involve decreased RANK expression on the OCL. Since the osteoclastogenesis studied in this system was driven in part by dexamethasone, these findings indicate that (+)-2-[1-(3-ethoxy-4-methoxyphenyl)-2-methanesulfonylethyl]-4-acetylaminoisoindolin-1,3-dione may be useful for counteracting the bone catabolic effects of corticosteroids.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of treating, preventing or managing bone loss, which comprises administering to a patient a therapeutically or prophylactically effective amount of (+)-2-[1-(3-ethoxy-4-methoxyphenyl)-2-methanesulfonylethyl]-4-acetylaminoisoindolin-1,3-dione: 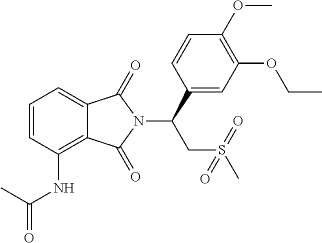 [SMILES: CCOc1cc([C@@H](CS(C)(=O)=O)N2C(=O)c3cccc(NC(C)=O)c3C2=O)ccc1OC]
or a pharmaceutically acceptable salt or solvate thereof.

2. The method of claim 1, wherein the bone loss is a primary bone loss.

3. The method of claim 2, wherein the primary bone loss is primary type 1 osteoporosis.

4. The method of claim 2, wherein the primary bone loss is primary type 2 osteoporosis.

5. The method of claim 1, wherein the bone loss is secondary to other underlying disorders.

6. The method of claim 5, wherein the underlying disorder is: an autoimmune disorder; a digestive or gastrointestinal disorder; a digestive or gastrointestinal disorder; a hematologic disorder; a neurological disorder; a mental illness; or cancer.

7. The method of claim 6, wherein the autoimmune disorder is rheumatoid arthritis, lupus, multiple sclerosis or ankylosing spondylitis.

8. The method of claim 6, wherein the digestive or gastrointestinal disorder is celiac disease or inflammatory bowel disease.

9. The method of claim 6, wherein the digestive or gastrointestinal disorder is diabetes, hyperparathyroidism, hyperthyroidism, Cushing's syndrome, thyrotoxicosis, premature menopause, or unusual testosterone levels.

10. The method claim 6, wherein the hematologic disorder is leukemia, lymphoma, multiple myeloma, sickle cell disease, a blood and bone marrow disorder or thalassemia.

11. The method of claim 6, wherein the neurological disorder is stroke, Parkinson's disease, multiple sclerosis or spinal cord injury.

12. The method of claim 6, wherein the mental illness is depression or an eating disorder.

13. The method of claim 6, wherein the cancer is bone, breast or prostate cancer;

14. The method of claim 1, wherein the bone loss is caused by a therapeutic agent.

15. The method of claim 14, wherein the therapeutic agent is a corticosteroid, an antiepileptic, L-thyroxine, an aromatase inhibitor, methotrexate, depot-progesterone, a gonadotropin-releasing hormone agonist, a proton pump inhibitor, a thiazolidinedione, or lithium.

16. The method of claim 15, wherein the corticosteroid is prednisone or dexamethasone.

17. The method of claim 15, wherein the antiepileptic is a barbiturate or phenytoin.

18. The method of claim 1, wherein the bone loss is osteogenesis imperfecta, osteomalacia, rickets, ostesis fibrosa cystic or Paget's disease.

19. The method of claim 1, wherein the (+)-2-[1-(3-ethoxy-4-methoxyphenyl)-2-methanesulfonylethyl]-4-acetylaminoisoindolin-1,3-dione or a pharmaceutically acceptable salt or solvate thereof, is administered in combination or alternation with a therapeutically effective amount of one or more additional active agents.

20. The method of claim 19, wherein the additional agent is a bisphosphonate, teriparatide, strontium renelate, raloxifene, denosumab, calcium, vitamin D, vitamin K or a combination thereof.
